(19) United States
(12) Patent Application Publication
(10) Pub. No.: US 2007/0155782 A1
(43) Pub. Date: Jul. 5, 2007

(54) Nk-1 receptor antagonists anesthesia recovery

(76) Inventors: Mary Anne Hickman, East Lyme, CT (US); Christine Miskell, Colchester, CT (US)

Correspondence Address: PHARMACIA & UPJOHN, 7000 Portage Road, KZO-300-104, KALAMAZOO, MI, 49001, US

(21) Appl. No.: 10/587,590

(22) PCT Filed: Jan. 6, 2005
(86) PCT No.: PCT/IB05/00010

§ 371 Date: Jul. 28, 2006

Related U.S. Application Data
(60) Provisional application No. 60/540,697, filed on Jan. 30, 2004.

(51) Int. Cl. A61K 31/4745

(52) U.S. Cl. 514/305

(57) ABSTRACT

The present invention is directed to the administration of a compound of the Formula (I) and (Ia), wherein R² is selected from the group consisting of methyl, ethyl, isopropyl, sec-butyl and tert-butyl, to an animal to improve anesthesia recovery

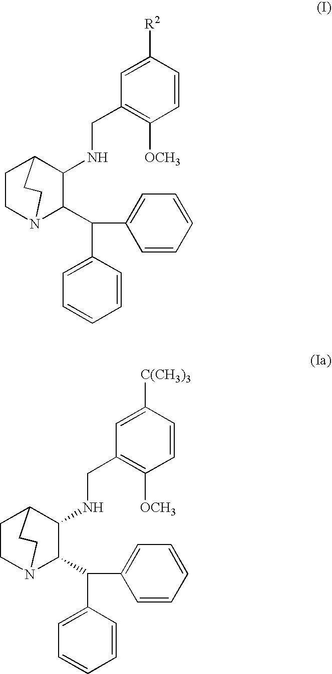

### FIELD OF INVENTION

The present invention relates to a method of improving anesthesia recovery comprising the step of administering to an animal a therapeutically effective amount of a pharmaceutical composition of a NK-1 receptor antagonist.

In particular, the present invention is directed to the administration of a compound of the Formula I, wherein R² is selected from the group consisting of methyl, ethyl, isopropyl, sec-butyl and tert-butyl, to an animal to improve anesthesia recovery.

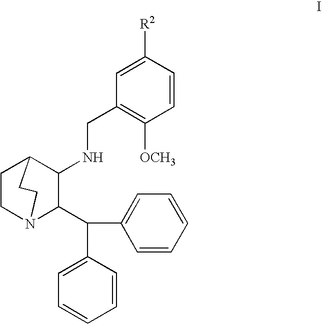

The invention is particularly directed to the administration of a compound of the Formula Ia to an animal to improve anesthesia recovery.

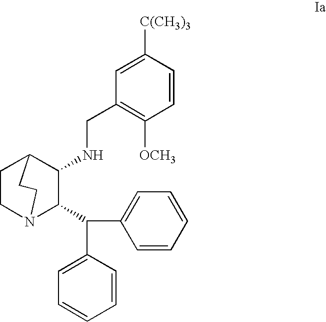

### BACKGROUND

The compounds of Formula I and Ia, NK-1 receptor antagonists, are effective as anti-emetic agents for mammals. Compounds of Formula I and Ia are the subject of U.S. Pat. No. 6,222,038 and U.S. Pat. No. 6,255,320. The preparation of the compounds are described therein. U.S. Pat. No. 5,393,762 also describes pharmaceutical compositions and treatment of emesis using NK-1 receptor antagonists. WO 03/009848 describes the use of NK-1 receptor antagonists to treat abnormal anxiety behaviour in companion animals. The text of the aforementioned patents and all other references cited in this specification are hereby incorporated by reference in their entirety.

Animals recovering after general anesthesia often appear dysphoric and exhibit behaviors such as excessive vocalization and purposeless movement. During the recovery period animals may experience traumatic injuries, especially to the head, in early attempts to achieve sternal recumbency and later while attempting to stand or walk too soon. This risk of injury is extremely high among horses, which despite specialized recovery stalls, frequently injure themselves and medical staff as they recover from anesthesia. Administration of an agent to an animal prior to induction of anesthesia, during an anesthetic episode or after anesthesia, that could improve the quality of the anesthetic recovery by reducing this potentially traumatic purposeless movement, would be valuable.

### SUMMARY OF INVENTION

In one aspect, the invention is directed to a method of improving anesthesia recovery comprising the step of administering to an animal in need of such treatment a therapeutically effective amount of a pharmaceutical composition of a NK-1 receptor antagonist; a pharmaceutically acceptable salt thereof, a prodrug of said compound or said salt, or a solvate or hydrate of said compound, said salt or said prodrug.

In a further aspect, the invention is directed to the use of a NK-1 receptor antagonist; a pharmaceutically acceptable salt thereof, a prodrug of said compound or said salt, or a solvate or hydrate of said compound, said salt or said prodrug, in the manufacture of a medicament for improving anesthesia recovery.

In one embodiment, the NK-1 receptor antagonist is a compound of Formula I 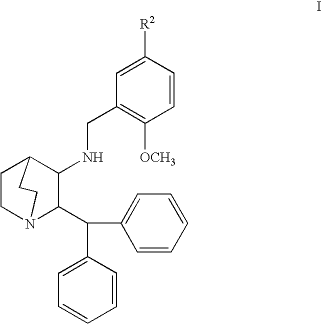
wherein R² is selected from the group consisting of methyl, ethyl, isopropyl, sec-butyl and tert-butyl, or a pharmaceutically acceptable salt thereof.

In a preferred embodiment, the compound of Formula I is a compound of Formula Ia, 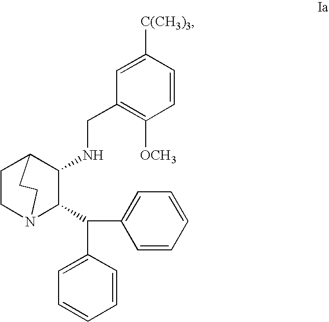
(2S,3S)-2-benzhydryl-N-(5-tert-butyl-2-methoxybenzyl)quinuclidin-3-amine, or a pharmaceutically acceptable salt thereof. In a more preferred embodiment, the compound is the citrate salt of the compound of Formula Ia, such as the citrate monohydrate salt.

In a preferred embodiment, the composition is parenterally, enterally or orally administered prior to, during or after an administration of a general anesthesia.

Preferentially, the composition is administered parenterally, with the pharmaceutical composition further comprising a pharmaceutically acceptable cyclodextrin. Preferentially, the cyclodextrin is β-cyclodextrin, hydroxypropyl β-cyclodextrin, sulfobutylether β-cyclodextrin or substituted cyclodextrins. In a preferred embodiment, the cyclodextrin is sulfobutylether β-cyclodextrin and the NK-1 receptor antagonist is (2S,3S)-2-benzhydryl-N-(5-tert-butyl-2-methoxybenzyl)quinuclidin-3-amine.

In a preferred embodiment, the composition further comprises a pharmaceutically acceptable preservative, preferably, meta-cresol.

In a further embodiment, the invention provides a pharmaceutical composition for improving anesthesia recovery comprising a NK-1 receptor antagonist; a pharmaceutically acceptable salt thereof, a prodrug of said compound or said salt, or a solvate or hydrate of said compound, said salt or said prodrug.

### Definitions

The term(s) “compound(s) of Formula I” and “compound(s) of this invention” as used herein, means a compound or compounds of Formula I, prodrugs thereof and pharmaceutically acceptable salts of the compounds or the prodrugs. The term “compound(s),” when referring to compounds of Formula I, also includes prodrugs of the compound(s) and pharmaceutically acceptable salts of the compound(s) or the prodrugs.

The term “neurokinin receptor antagonist” as used herein includes, but is not limited to, compound of Formula I or various ligands, compounds, and/or substances that can specifically bind to the NK-1 neurokinin receptor and includes, but are not limited to, piperazine compounds, spiro-substituted azacycles, dialkyline piperadino compounds, trypthophan urea, polycyclic amine compounds, substituted arylaliphatic compounds, aromatic amine compounds, quaternary ammonium salts or aromatic amine compounds, aryl substituted hetrocycles, polycyclicamine compounds, substituted aryl piperazines, carboxamide derivatives, bis-piperadinyl non-peptidal compounds, salts thereof, and any other similar neurokinin receptor antagonist known to those of skill in the art.

“Improving anesthesia recovery” as used herein includes improving the quality of anesthetic recovery by reducing excessive vocalization and/or purposeless movement (including achieving sternal recumbency and attempts to stand and walk too soon).

The term “cyclodextrin” as used herein means a cyclic oligosaccharide. Cyclodextrins typically vary in shape and size, but define a hydrophobic cavity and can form inclusion compounds with other organic molecules, with salts, and with halogens either in solid state or in aqueous solution. Methods for preparing cyclodextrins are well known to those of skill in the art and many cyclodextrins are commercially available. There are three main types of cyclodextrins: α-cyclodextrin, β-cyclodextrin and γ-cyclodextrin. The term “cyclodextrin” also includes various substituted cyclodextrins, including as side chains any organic moiety or a heteroorganic moiety. Substituted cyclodextrins also include cyclodextrins that have been alkylated, hydroxyalkylated, or reacted to form a sulfoalkyl ether.

As used herein, cyclodextrins and/or substituted cyclodextrins include, but are not limited to, sulfobutylether cyclodextrin, hydroxypropyl cyclodextrin, hydroxyethyl cyclodextrin, glucosyl cyclodextrin, maltosyl cyclodextrin, hydroxypropyl-β-cyclodextrin, sulfobutylether-β-cyclodextrin, hydroxyethyl-β-cyclodextrin, hydroxypropyl-γ-cyclodextrin, hydroxyethyl-β-cyclodextrin, dihydroxypropyl-β-cyclodextrin, glucosyl-β-cyclodextrin, diglycosyl-β-cyclodextrin, maltosyl-β-cyclodextrin, maltosyl-γ-cyclodextrin, maltotrialsyl-β-cyclodextrin, maltotrialsyl-γ-cyclodextrin, dimaltosyl-β-cyclodextrin, cyclodextrin derivatives, various mixtures of cyclodextrin derivatives thereof, mixtures such as maltosyl-β-cyclodextrin/dimaltosyl-β-cyclodextrin, and any other similar cyclodextrin known to those of skill in the art.

The term “mammals” or “animals,” as used herein, refers to humans, companion animals (e.g., dogs, cats and horses, especially dogs), food-source animals (e.g., cows, pigs and sheep), zoo animals and other similar animal species.

The term “therapeutically effective amount” means an amount of a compound of the present invention that (i) treats or prevents the particular condition or disorder, (ii) attenuates, ameliorates or eliminates one or more symptoms of the particular condition or disorder, or (iii) prevents or delays the onset of one or more symptoms of the particular condition or disorder described herein.

The term “pharmaceutically acceptable” indicates that the substance or composition must be compatible chemically and/or toxicologically, with the other ingredients comprising a formulation, and/or the mammal being treated therewith.

The terms “treating”, “treat”, or “treatment” embrace both palliative and preventative, i.e., prophylatic, treatment.

### BRIEF DESCRIPTION OF THE DRAWINGS

Other advantages of the present invention will be readily appreciated, as the same becomes better understood by reference to the following detailed description when considered in connection with the accompanied drawing, wherein:

FIG. 1 is a chart illustrating the effect of the compound of Formula Ia on the quality of anesthetic recovery in dogs.

### DETAILED DESCRIPTION

The present invention provides for a method of improving anesthesia recovery in patients by administering a NK-1 antagonist prior to, during or after the administration of general anesthesia. In particular, the invention is directed to administering a compound of Formula I or Ia prior to, during or after the administration of general anesthesia to improve anesthesia recovery in patients. If administering the compound of Formula I or Ia after general anesthesia, it is preferentially administered within about thirty (30) minutes during the recovery stage.

The compound of Formula I and Ia can be prepared as described in U.S. Pat. No. 6,222,038 or U.S. Pat. No. 6,255,038. Salts of the compound of Formula I or Ia, in particular the citrate salt, can be prepared as described in the above patents. Alternatively, the compound of Formula I can also be prepared as described in co-pending U.S. provisional application, No. 60/541,323 assigned to and owned by Pfizer, Inc.

One possible preparation of the crystalline citrate monohydrate salt of the compound of Formula Ia is by suspending 47 grams of the free base in 470 mL of isopropyl ether under ambient conditions. To the slurry was added 21.42 grams anhydrous citric acid at room temperature. The mixture was converted to the monohydrate by suspending in 150 mL of water for eighteen hours and filtered, providing a white crystalline solid.

Injectable formulations may be prepared by dissolving a therapeutically effective amount of the compound of Formula I or Ia in an aqueous pharmaceutically acceptable diluent. A pharmaceutically acceptable salt of the compound of Formula I or Ia may also be used, such as the citrate or malate salts. A cyclodextrin may be added to the solution in a concentration range of about 2% to about 40%. Preferably, the cyclodextrin comprises about 5% to about 20% of the pharmaceutical composition and more preferably about 5% to about 10%. Pharmaceutical compositions comprising the compound of I or Ia, cyclodextrin and a pharmaceutically acceptable preservative are described in co-pending U.S. provisional application No. 60/540,897 assigned to and owned by Pfizer, Inc. A method of improving injection site toleration of the compound of Formula I or Ia, as well as pharmaceutical compositions, is described in co-pending U.S. provisional application No. 60/540,644 assigned to and owned by Pfizer, Inc. The above applications are hereby incorporated in their entirety for all purposes.

As used herein, a “therapeutically effective amount” for a dosage unit, for the purposes of the present invention, may typically be about 0.5 mg to about 500 mg of active ingredient. The dose may vary, however, depending on the species, variety, etc. of animal to be treated, the disease severity, the body weight of the animal and the route of administration. Accordingly, based upon body weight, typical dose ranges of the active ingredient may be from about 0.01 to about 100 mg per kg of body weight of the animal. Preferably, the range is from about 0.10 mg to about 10 mg per kg of body weight.

The veterinary practitioner, or one skilled in the art, will be able to determine the dosage suitable for the particular individual patient, which may vary with the species, age, weight, response of the particular patient and route of administration. The above dosages are exemplary of the average case. Accordingly, higher or lower dosage ranges may be warranted, depending upon the above factors, and are within the scope of this invention.

According to the methods of the invention, when a combination of the compound of Formula I or Ia and at least one other pharmaceutical agent are administered together, such administration can be sequential in time or simultaneous, with sequential being preferred. For sequential administration, the compound of Formula I or Ia and the additional pharmaceutical agent can be administered in any order. For example, the compound of Formula I or Ia may be administered prior to, during or after preanesthetic administration. When the compound of Formula I or Ia and the additional pharmaceutical agent are administered sequentially, the administration of each can be by the same or different methods.

According to the methods of the invention, a compound of the present invention or a combination of a compound of Formula I or Ia and at least one additional pharmaceutical agent (referred to herein as a “combination”) is preferably administered in the form of a pharmaceutical composition. Accordingly, a composition of the compound of Formula I or Ia can be administered to a patient by various means, including orally, buccally, nasally and parenterally (e.g. intravenous, intramuscular or subcutaneous).

Compositions suitable for parenteral injection generally include pharmaceutically acceptable sterile aqueous or nonaqueous solutions, dispersions, suspensions, or emulsions, and sterile powders for reconstitution into sterile injectable solutions or dispersions. Examples of suitable aqueous and nonaqueous carriers or diluents (including solvents and vehicles) include water, ethanol, polyols (propylene glycol, polyethylene glycol, glycerol, and the like), suitable mixtures thereof, vegetable oils (such as olive oils) and injectable organic esters, such as ethyl oleate. Proper fluidity can be maintained, for example, by the use of a coating such as lecithin, by the maintenance of the required particle size in the case of dispersions, and by the use of surfactants. Prevention of microorganism contamination of the compositions can be accomplished with various antibacterial and antifungal agents.

Solid dosage forms for oral administration include capsules, tablets, powders and granules. In such solid dosage forms, a compound of the present invention or a combination is admixed with at least one inert customary pharmaceutical excipient (or carrier) such as sodium citrate or dicalcium phosphate or (a) fillers or extenders (e.g. starches, lactose, sucrose, mannitol, silicic acid and the like); (b) binders (e.g. carboxymethylcellulose, alginates, gelatin, polyvinyl pyrrolidone, sucrose, acacia and the like); (c) humectants (e.g. glycerol and the like); (d) disintegrating agents (e.g. agar-agar, calcium cargonate, potato or tapioca starch, alginic acid, certain complex silicates, sodium carbonate and the like); (e) solution retarders (e.g., paraffin and the like); (f) absorption accelerators (e.g., quaternary ammonium compounds and the like); (g) wetting agents (e.g., cetyl alcohol, glycerol monostearate and the like); (h) adsorbents (e.g., kaolin, bentonite and the like); and/or (i) lubricants (e.g., talc, calcium stearate, magnesium stearate, solid polyethylene glycols, sodium lauryl sulfate and the like). In the case of capsules and tablets, the dosage forms may also comprise buffering agents.

Solid compositions of a similar type may also be used as fillers in soft or hard filled gelatin capsules using such excipients as lactose or milk sugar, as well as high molecular weight polyethylene glycols, and the like.

Solid dosage forms such as tablets, dragees, capsules, and granules can be prepared with coatings and shells, such as enteric coatings and others well known in the art. They may also contain opacifying agents, and can also be of such composition that they release the compound of the present invention and/or the additional pharmaceutical agent in a delayed manner. Examples of embedding compositions that can be used are polymeric substances and waxes. The drug can also be in micro-encapsulated form, if appropriate, with one or more of the above-mentioned excipients.

Liquid dosage forms for oral administration include pharmaceutically acceptable emulsions, solutions, suspensions, syrups, and elixirs. In addition to the compound of the present invention or the combination, the liquid dosage form may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed oil, groundnut oil, corn germ oil, olive oil, castor oil, sesame seed oil and the like), glycerol, tetrahydrofurfuryl alcohol, polyethylene glycols and fatty acid esters of sorbitan, or mixtures of these substances, and the like.

Besides such inert diluents, the composition can also include excipients, such as wetting agents, emulsifying and suspending agents, sweetening, flavoring, and perfuming agents.

Suspensions, in addition to the compound of the present invention or the combination, may further comprise suspending agents, e.g., ethoxylated isostearyl alcohols, polyoxyethylene sorbitol and sorbitan esters, microcrystalline cellulose, aluminum metahydroxide, bentonite, agar-agar, and tragacanth, or mixtures of these substances, and the like.

### EXPERIMENTALS

The patients utilized in the study were maintained in standard facilities with appropriate heating and ventilation. All dogs were placed under general anesthesia. The duration of anesthesia, time to extubation and time to sternal recumbency were recorded. Also noted were behaviors indicative of dysphoria such as vocalization and purposeless movement. A visual analog scale (“VAS”) was used to score the “quality” of the entire recovery period.

As used herein, the term “duration of anesthesia” is the time in minutes elapsed between intubation and cessation of anesthetic gases.

As used herein, the term “time to extubation” is the time in minutes elapsed between cessation of anesthetic gases and the need for removal of the endotracheal tube due to spontaneous swallowing.

As used herein, the term “time to Sternal Recumbency” is the time in minutes elapsed between cessation of anesthetic gases and the time the dog could physically place and maintain itself in sternal recumbency.

Evaluation of Recovery Period. A visual analog scale (“VAS”) was used to score the “quality” of the recovery period (0=smooth recovery, 10=rough recovery). Factors considered included, but were not limited to, vocalization, purposeless movement as well as the subjective degree of dysphoria experienced by the dog. The study director, who was blinded to treatment assignments, assigned recovery scores.

Statistical Analysis. Data from two trials conducted according to the above procedure were combined for analysis purposes. The data in the following Tables and Figures are mean±standard deviation. Duration of anesthesia, time to extubation and time to sternal recumbency data were analyzed using a paired t-test for means in Excel. Recovery VAS data were analyzed using SAS Statistical Software.

### Experiment A: 1.0 mg/kg Dose of Compound of Formula Ia With Saline Control

Experimental mature mongrel dogs, both male and female, weighing about 16-26 kilograms, were subcutaneously administered 1.0 mg/kg compound of Formula Ia one hour prior to preanesthetic administration. Control dogs were subcutaneously administered an equivalent volume of saline solution one hour prior to preanesthetic administration. The duration of anesthesia, time to extubation and time to sternal recumbency were recorded. The patients were monitored continuously during the anesthetic and recovery periods.

Preparation of 1.0 mg/kg compound of Formula Ia dose: Formulations were prepared by dissolving the compound of Formula Ia (10 mg/mL) and SBE-CD (10%) in distilled water to form a solution. The solution was sonicated to facilitate complete dissolution and filtered through a 0.22 μm Millipore syringe top filter prior to injection.

Administration of Dose: Solutions of compound of Formula Ia (1.0 mg/kg) or saline placebo (0.1 mL/kg) were administered by subcutaneous injection (“SC”).

Anesthetic Protocol: General anesthesia was induced using the following protocol: (1) One hour prior to preanesthetic administration, the patients were treated with subcutaneous administration of either a dose of compound of Formula Ia, as prepared above, or the saline control (0.1 mL/kg).

(2) Thirty (30) minutes prior to induction (preanesthetic), the patients were administered glycopyrrolate (0.01 mg/kg SC) and butorphanol (0.1 mg/kg SC).

(3) During the induction period, the patients were administered intravenous methohexital (8 mg/kg IV).

(4) The patients were then maintained with inhaled isoflurane at concentrations ranging from 0.5 to 3%.

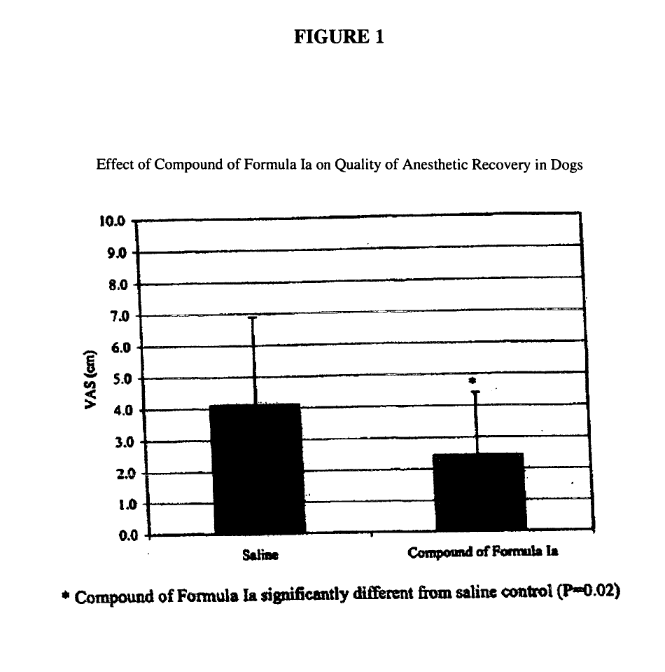

Results and Discussion. Duration of anesthesia, time to extubation and time to sternal recumbency were similar for the compound Ia and saline groups (Table 1). Dogs that received the compound Ia dose received significantly lower recovery scores than those dogs administered the saline dose (p=0.02, Table 1, FIG. 1). Recovery VAS scores for dogs administered the compound Ia dose were 43.9% lower than saline controls. The results of this study indicate that administration of compound Ia prior to anesthesia improved the quality of anesthetic recovery in dogs. Those dogs that were administered with the compound of Formula Ia dose exhibited less vocalization, less purposeless movement and less ataxia upon standing than saline controls.

TABLE I
Effect of Compound of Formula Ia on Various Parameters Related to Anesthetic Recovery

| | Saline (0.1 mL/kg SC) | | | | Compound of Formula Ia (1 mg/kg SC) | | | |
|---|---|---|---|---|---|---|---|---|
| | | | Time to | | | | Time to | |
| | Duration of | Time to | Sternal | Recovery | Duration of | Time to | Sternal | Recovery |
| | Anesthesia | Extubation | Recumbency | Quality | Anesthesia | Extubation | Recumbency | Quality |
| Dog ID | (min) | (min) | (min) | (cm) | (min) | (min) | (min) | (cm) |
| 30467 | 30 | 2 | 5 | 2.4 | 30 | 1 | 3 | 2.4 |
| 00345 | 28 | 1 | 3 | 8.7 | 30 | 2 | 4 | 7.1 |
| 00368 | 29 | 1 | 8 | 1.4 | 30 | 2 | 3 | 1.4 |
| 30390 | 30 | 4 | 6 | 2.2 | 30 | 2 | 6 | 2.1 |
| 227641 | 30 | 3 | 5 | 4.2 | 29 | 1 | 3 | 0.7 |
| 226998 | 30 | 1 | 2 | 5.3 | 30 | 3 | 3 | 0.9 |
| 60791 | 30 | 1 | 11 | 5.4 | 34 | 5 | 1 | 3.9 |
| MEE | 30 | 7 | 14 | 0.6 | 30 | 4 | 11 | 0.2 |
| MJG | 30 | 4 | 12 | 0.8 | 30 | 4 | 9 | 1.1 |
| MES | 30 | 3 | 5 | 3.3 | 30 | 5 | 7 | 1.8 |
| MFU | 28 | 1 | 5 | 7.8 | 30 | 2 | 9 | 1.9 |
| MHI | 30 | 1 | 3 | 7.2 | 30 | 5 | 6 | 4.7 |
| Mean ± SD | 30 ± 1 | 2 ± 2 | 7 ± 4 | 4.1 ± 2.8 | 30 ± 1 | 3 ± 2 | 5 ± 3 | 2.3 ± 2 * |
| p vs saline | N/A | N/A | N/A | N/A | 0.1 | 0.4 | 0.3 | 0.02 |

* significantly different from saline control, p < 0.05

### Experiment B: 0.5 mg/kg Dose of Compound of Formula Ia

Fifteen experimental geriatric beagles, weighing 9-16 kilograms and undergoing anesthesia for dental prophylaxis, were used in this study. All dogs were fasted overnight prior to anesthesia. Experimental dogs were subcutaneously administered 0.5 mg/kg compound of Formula Ia at the time of pre-anesthetic medication. Control dogs received no treatment in addition to routine pre-anesthesia medications. The duration of anesthesia, time to extubation and time to sternal recumbency were recorded.

Preparation of 0.5 mg/kg compound of Formula Ia dose: Formulations were prepared by dissolving the compound of Formula Ia (10 mg/mL) and SBE-CD (10%) in distilled water to form a solution. The solution was sonicated to facilitate complete dissolution and filtered through a 0.22 μm Millipore syringe top filter prior to injection.

Administration of Dose: Solutions of compound of Formula Ia (0.5 mg/kg) were administered by subcutaneous injection.

Anesthetic Protocol: General anesthesia was induced and maintained using the following protocol:

(1) Preanesthetic administration: the Experimental patients were treated with subcutaneous administration of a dose of compound of Formula Ia, as prepared above. Both experimental and control patients were administered glycopyrrolate (0.01 mg/kg SC), butorphanol (0.1 mg/kg SC) and penicillin (30,000 units/kg SC)

(2) During the induction period, the patients were administered intravenous methohexital (8 mg/kg IV).

(3) The patients were then maintained with inhalant isoflurane at concentrations ranging from 0.5 to 3%.

The patients were monitored continuously during the anesthetic and recovery periods.

Results and Discussion. There was no statistical difference between the experimental and control groups in terms of duration of anesthesia, time to extubation, time to sternal recumbency, or visual analog score for “smoothness” of recovery period (Table 2). Subjectively, dogs treated with compound of Formula Ia (0.5 mg/kg SC), during the preoperative period tended to look less anxious during the recovery period than control dogs. Treated dogs tended to receive lower visual analog scores for the smoothness of the recovery period than control dogs. All treated dogs (7 out of 7) received visual analog scores of less than 5 cm while only 50% of control dogs (4 out of 8) received a visual analog score of less than 5 cm.

TABLE 2
Effect of Preoperative Administration of Compound of Formula Ia (0.5
mg/kg SC) on Recovery from General Anesthesia in Beagle Dogs

| | Interval of | | Interval of | | Time to | |
|---|---|---|---|---|---|---|
| | Premedication to | Duration of | Premedication to | Time to | Sternal | |
| | Induction | Anesthesia | End Anesthesia | Extubation | Recumbency | Recovery Score |
| | Mean ± SD | Mean ± SD | Mean ± SD | Mean ± SD | Mean ± SD | Mean ± SD |
| | (min) | (min) | (min) | (min) | (min) | (cm) |
| Compound of | 60.14 ± 39.63 | 73.43 ± 17.97 | 133.57 ± 35.48 | 5.00 ± 4.47 | 10.86 ± 10.67 | 3.46 ± 1.54 |
| Formula I | | | | | | |
| 0.5 mg/kg SC | | | | | | |
| Control | 84.25 ± 54.61 | 69.75 ± 12.19 | 154.00 ± 55.37 | 5.13 ± 3.27 | 11.13 ± 4.45 | 4.59 ± 2.24 |

1-10. (canceled)

11. A method of improving anesthesia recovery comprising the step of administering to an animal in need of such treatment a therapeutically effective amount of a pharmaceutical composition of a NK-1 receptor antagonist; a pharmaceutically acceptable salt thereof, a prodrug of said compound or said salt, or a solvate or hydrate of said compound, said salt or said prodrug.

12. The use of a composition comprising a NK-1 receptor antagonist; a pharmaceutically acceptable salt thereof, a prodrug of said compound or said salt, or a solvate or hydrate of said compound, said salt or said prodrug, in the manufacture of a medicament for improving anesthesia.

13. A method of using an NK-1 receptor antagonist; a pharmaceutically acceptable salt thereof; a prodrug of said compound or said salt, or a solvate or hydrate of said compound, said salt or said prodrug, for improving the quality of anesthetic recovery by reducing excessive vocalization and/or purposeless movement.

14. A method or use according to claim 13 wherein the NK-1 receptor antagonist is a compound comprising Formula I 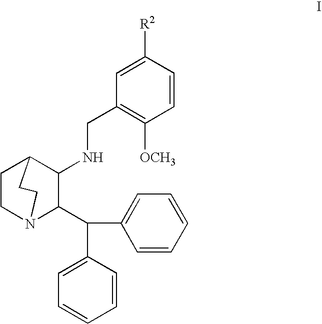
wherein R² is selected from the group consisting of methyl, ethyl, isopropyl, sec-butyl and tert-butyl, or a pharmaceutically acceptable salt thereof.

15. A method or use according to claim 14 wherein said compound comprises Formula Ia, 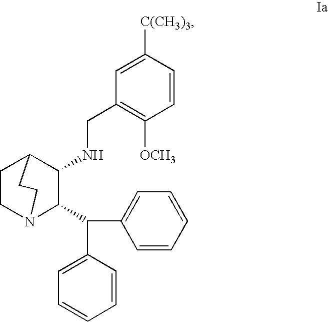
(2S,3S)-2-benzhydryl-N-(5-tert-butyl-2-methoxybenzyl)quinuclidin-3-amine, or a pharmaceutically acceptable salt thereof.

16. The method or use according to claim 15 wherein said compound is the citrate salt of the compound of Formula Ia.

17. The method or use according to claim 12 wherein said composition is parenterally, enterally or orally administered prior, during or after an administration of a general anesthesia.

18. The method or use according to claim 17 wherein said composition is administered parenterally.

19. The method or use according to claim 18 wherein said composition further comprises a pharmaceutically acceptable cyclodextrin.

20. The method or use according to claim 18 wherein the amount of the NK-1 antagonist is 0.01 mg/kg to 100 mg/kg of a patient's body weight.

21. The method or use according to claim 19 wherein the amount of the NK-1 antagonist is 0.01 mg/kg to 100 mg/kg of a patient's body weight.

22. A pharmaceutical composition for improving anesthesia recovery comprising: a NK-1 receptor antagonist; a pharmaceutically acceptable salt thereof, a prodrug of said compound or said salt, or a solvate or hydrate of said compound, said salt or said prodrug.

23. A pharmaceutical composition of claim 22 where the NK-1 receptor is a compound comprising Formula I wherein R² is selected from the group consisting of methyl, ethyl, isopropyl, sec-butyl and tert-butyl, or a pharmaceutically acceptable salt thereof.

24. A pharmaceutical composition of claim 23 where the NK-1 receptor is a composition comprising, (2S,3S)-2-benzhydryl-N-(5-tert-butyl-2-methoxybenzyl)quinuclidin-3-amine, or a pharmaceutically acceptable salt thereof.
